(19) United States
(12) Patent Application Publication
(10) Pub. No.: US 2016/0310397 A1
(43) Pub. Date: Oct. 27, 2016

(54) METHOD OF ACHIEVING TARGETED DELIVERY OF A SCALP CLEANSING COMPOSITION AND A CONDITIONING SHAMPOO COMPOSITION

(71) Applicant: The Procter & Gamble Company, Cincinnati, OH (US)

(72) Inventors: Eric Scott Johnson, Hamilton, OH (US); Michael Frederick Niebauer, Singapore (SG); Robert Wayne Glenn, JR., Liberty Township, OH (US); Jazmin Veronica Torres Rivera, Cincinnati, OH (US)

(21) Appl. No.: 15/136,020

(22) Filed: Apr. 22, 2016

Related U.S. Application Data
(60) Provisional application No. 62/151,710, filed on Apr. 23, 2015.

(51) Int. Cl. A61K 8/58; A61Q 5/02; A61Q 5/12; A61K 8/46; A61K 8/42; A61K 8/44; A61K 8/37; A61K 8/81; A61K 8/97; A61K 8/27; A61K 8/34; A61K 8/49; A61K 8/891; A61K 8/73; A61Q 5/00; A45D 34/04; A45D 19/02; A61K 8/04

(52) U.S. Cl. CPC A61K 8/58; A61K 8/046; A61Q 5/02; A61Q 5/12; A61K 8/463; A61K 8/42; A61K 8/44; A61K 8/37; A61K 8/817; A61K 8/97; A61K 8/585; A61K 8/27; A61K 8/342; A61K 8/34; A61K 8/49; A61K 8/891; A61K 8/731; A61Q 5/006; A45D 34/042; A45D 19/02; A61K 2800/87; A61K 2800/884; A45D 2019/0033

(57) ABSTRACT

A method of delivering a scalp cleansing composition to the scalp and a conditioning shampoo composition to the rest of the hair directed to: Applying a scalp cleansing composition directly onto the scalp using an applicator wherein the scalp cleansing composition comprises a sensate; Applying a conditioning shampoo composition on the length and ends of the hair, where length is defined as the hair starting below the root line at the back of the neck/base of the head and the ends are defined as the 4 cm of the hair furthest away from the scalp; wherein % increase scalp deposition is 10%.

### FIELD OF INVENTION

The present invention is directed to a method of achieving targeted delivery of an antidandruff composition to the scalp and a cosmetic hair composition to the hair.

### BACKGROUND OF THE INVENTION

Getting the scalp and hair into their optimal condition requires different technical solutions due to their unique needs and starting conditions. The conventional approach is an application of a single composition comprising a detersive surfactant, anti-dandruff active and a conditioning agent such as a silicone, hydrocarbon oil, fatty ester, and other conventional materials in a shampoo composition.

A typical starting condition of the scalp can be itchy, flaky, not clean feeling, and irritated. Today, conventional antidandruff compositions provide benefits by depositing scalp health actives on the scalp to treat these conditions.

### Targeted Rationale

Targeting specific actives to the scalp and other actives to the hair allow for more consumer meaningful, noticeable and delightful experiences. This approach yields an increase in scalp active retention during the rinsing process which can lead to an improvement in consumer self-assessment of scalp attributes (itch reduction, flake reduction, greasy feel reduction, etc.). In addition, providing more targeted deposition of conditioning actives to the hair can lead to an improvement in consumer self-assessment of hair smoothness attributes.

Anti-dandruff particles, like zinc pyrithione and selenium sulphide, can become trapped between hair fibres during typical shampoo washing procedures and therefore do not effectively reach the scalp i.e. lower on-scalp deposition and hence cannot so effectively deliver the benefit. Coacervates are often used to provide superior wet conditioning and conditioning active delivery to the hair from shampoos. Coupling anti-dandruff active delivery and coacervate enables deposition from a shampoo and can result in particle agglomeration causing even more anti-dandruff particles to get trapped in the hair. Using a targeted applicator for the anti-dandruff shampoo will enable particles to be pushed through the hair to more effectively deliver active to the scalp while also using a high conditioning shampoo on the longer parts of the hair can provide superior coacervate enabled conditioning and conditioning active delivery.

### Consumer Unmet Need

Getting the scalp and hair into their optimal condition requires different technical solutions due to their unique needs and starting conditions. The conventional approach is an application of a single composition comprising a detersive surfactant, anti-dandruff active and a conditioning agent such as a silicone, hydrocarbon oil, fatty ester, and other conventional materials in a shampoo composition.

A typical starting condition of the scalp can be itchy, flaky, not clean feeling, and irritated. Today, conventional antidandruff compositions provide benefits by depositing scalp health actives on the scalp to treat these conditions.

The tables below are consumer survey results related to assessment of scalp conditions and to consumer concerns about scalp conditions.

Scalp Conditions Consumers are Concerned About in Past 4 Weeks (n=4282)

| | |
|---|---|
| Scalp Itching | 33% |
| Visible Flakes (hair/clothing) | 18% |
| Dandruff When Scratch/Comb | 14% |
| Scalp Doesn't Stay Clean | 14% |
| Scalp Irritation/Sensitivity | 13% |
| Scalp Odor | 10% |
| Product Build-Up on Scalp | 7% |
| Very Shiny Scalp | 5% |
| Scalp Tightness | 5% |
| None of the Above | 48% |

However the conventional approach also deposits the same materials on the hair, which can leave the user perceive their hair is dry.
Anti-Dandruff Consumers Experienced Dry Hair in Past 4 Weeks (n=1530)

| | |
|---|---|
| Hair is Dry | 27% |

The starting condition of the hair can be dry, shed, look dull, be damaged, etc. In addition hair strand conditions vary from near the scalp where it is more virgin to the ends of the hair where it can be more damaged due to mechanical, environmental, and/or chemical damage.
Hair Conditions Consumers are Concerned About in Past 4 Weeks (n=4282)

| | |
|---|---|
| Hair is Dry | 24% |
| Hair Shedding At Roots | 22% |
| Dull Looking Hair | 22% |
| Damage From Perming, Coloring, Sun, Environment, Hot Styling | 22% |
| Implements | |
| Not Enough Volume | 19% |
| Hair Breakage at Tips | 17% |
| Hair is Frizzy | 17% |
| Color Fading, Unevenness, Brassiness | 17% |
| Split Ends | 15% |
| Excess Oil at Roots | 15% |
| Difficulty in Maintaining Style | 14% |
| Hair Does Not Stay Clean Long | 14% |
| Flyaway Hair/Static Hair | 13% |
| Hair Tangled/Knots | 13% |
| Hair Reacts Badly to Humidity | 13% |
| Texture of Grey Hair | 10% |
| Stiff/Brittle Hair | 9% |
| Lack of Hair Movement | 9% |
| Attracting Environmental Odors | 9% |
| Too Much Volume | 8% |
| Product Build-Up | 6% |
| Hair/Ends Feeling Stripped | 6% |
| None of the above | 26% |

Today, we solve this by depositing conditioning agents, within the same composition as an anti-dandruff material, along the length of the hair to treat these conditions. However the conventional approach also deposits the same materials on the scalp, which can leave the scalp feeling oily or greasy.

Anti-Dandruff Consumers Experienced an Oily Scalp in Past 4 Weeks (n=1530)

| | |
|---|---|
| Scalp is Oily | 31% |

Thus there is a need to identify ways to predominantly target scalp actives to the scalp and hair actives to the hair allowing for faster resolution of scalp and hair conditions while reducing the amount of cross-deposits.

### SUMMARY OF INVENTION

In an embodiment of the present invention, it is directed to method of delivering a scalp cleansing composition to the scalp and a conditioning shampoo composition to the rest of the hair comprising: Applying a scalp cleansing composition directly onto the scalp using an applicator wherein the scalp cleansing composition comprises a sensate; Applying a conditioning shampoo composition on the length and ends of the hair, where length is defined as the hair starting below the root line at the back of the neck/base of the head and the ends are defined as the 4 cm of the hair furthest away from the scalp; wherein % increase scalp deposition is 10%.

### DETAILED DESCRIPTION OF THE INVENTION

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography “QS” means sufficient quantity for 100%.

All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Herein, “comprising” means that other steps and other ingredients which do not affect the end result can be added. This term encompasses the terms “consisting of” and “consisting essentially of”. The compositions, methods, uses, kits, and processes of the present invention can comprise, consist of, and consist essentially of the elements and limitations of the invention described herein, as well as any of the additional or optional ingredients, components, steps, or limitations described herein.

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the hair care composition.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. A polymer comprises at least two monomers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

“Kit,” as used herein, means a packaging unit comprising a plurality of components. An example of a kit is, for example, a first composition and a separately packaged second composition. Another kit may comprise a first composition and an energy delivery device. A different kit may comprise three different types of separately packaged composition and a hair styling implement. A further kit may comprise application instructions comprising a method and a composition/formulation.

The term “coacervate” as used herein, means the complex which forms between surfactant and polymer that may either be soluble or insoluble in the neat composition, typically forming an insoluble complex in the neat composition, and which may become less soluble upon dilution and thus yielding an increase in its level of phase separation or precipitate in solution.

### Active Deposition Protocol

The on-scalp deposition of the scalp active is at least about 0.5 microgram/cm². The on-scalp deposition of the scalp active is important in view of ensuring that the scalp active reaches the scalp where it is able to perform its function. In an embodiment, the deposition of the scalp active on the scalp is at least about 1 microgram/cm², or at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the scalp active is measured by having the hair of individuals washed with a composition comprising an scalp active, for example a composition pursuant to the present invention, by trained cosmetician according to protocol outlined in this present invention or a conventional washing protocol. The hair is then parted on an area of the scalp to allow an open-ended glass cylinder to be held on the surface while an aliquot of an extraction solution, consisting of a EDTA, ethanol, isopropyl alcohol, water or a combination thereof, is added and agitated prior to recovery. Analytical determination of scalp active content by conventional methodology, such as HPLC (for pyrithione) and ICP (for total Zinc and Se).

In an embodiment, the on-scalp deposition of zinc pyrithione is at least about 1 microgram/cm², basic zinc carbonate is at least about 1 microgram/cm², menthol is about 0.2 microgram/cm² and SeS is about 0.8 microgram/cm².

In an embodiment of the present invention, the % increase scalp disposition is the deposition of the scalp active achieved by the targeted delivery method of the present invention minus the deposition of scalp active achieved by a conventional application of the scalp cleansing composition and that difference being divided by the deposition of scalp active achieved by such conventional application. In an embodiment of the present invention, the % increase scalp deposition is at least 10%, in an embodiment at least 20%, or in a further embodiment is at least 50%, and in a further embodiment at least 100%.

### Viscosities

a. Cleansing Product—In the present invention, for a cleansing composition, the viscosity may be thin enough that it easily dispenses from the package and distributes through the scalp and thick enough to stay at the root without dripping from hair as well as hands. One means to achieve excellent deep and uniform cleansing on in such a way that rheological parameters such as viscosity are low. However, such compositions would not stay where applied and would drip, run, or transfer to other areas in a manner such as to reduce its effectiveness. Thus, it has heretofore been believed that an adequate viscosity of a cleansing composition is necessary to retain cleansing composition in the defined locations as to deliver benefits.
   i. Liquid Composition Dispensing Using Hands without an Applicator:
      The composition has a zero shear viscosity value measured at 0.01 Pa of from about 1,000 Pa s to about 20,000 Pa s, in an embodiment greater from about 1,500 Pa s to about 15,000 Pa s, in a further embodiment from about 2,000 Pa s to about 10,000 Pa s.
         In a further embodiment the composition has a zero shear viscosity measured at 0.01 Pa of less than about 20,000 Pa s; in a further embodiment, less than about 15,000 Pa s; in yet a further embodiment less than about 10,000 Pa s.
         Thin enough to cover and spread on scalp, thick enough to not drip from hands when apply.
   ii. Liquid Composition Dispensing with an Applicator (Non-Foaming)
      The composition is shear thinning and has (1) a zero shear viscosity value measured at 0.01 Pa of from about 1,500 Pa s to about 10,000 Pa s, in an embodiment from about 2,000 Pa s to about 8,000 Pa s, and in a further embodiment from about 2,500 Pa s to about 6,000 Pa s; (2) a moderate stress viscosity value measured at 1 Pa viscosity of greater than about 500 Pa s and less than 5,000 Ps, in an embodiment from about 600 Pa s to about 4,500 Pa s, in a further embodiment from about 700 Pa s to about 4,000 Pa s; and (3) a high shear rate viscosity value measured at 100 l/s of less than about 100 Pa s, in an embodiment less than about 70 Pa s, and in a further embodiment less than about 50 Pa s.
         Shear thinning through the applicator for easy of dispensing
Foam enables low viscosity uniform spreading, lathering and coverage on scalp and through the hair, not dripping and wherein viscosity is low enough for foaming.
   iii. Liquid Composition Dispensing with a Foam Applicator
      The composition has a zero shear viscosity value measured at 0.01 Pa of less than about 15,000 Pa s, in an embodiment less than about 10,000 Pa s, in a further embodiment less than about 8,000 Pa S.
b. Conditioning Product—In the present invention, the viscosity of a conditioning product may be thin enough that it easily dispenses from the package while thick enough that it does not drip from the hair.
   One means to achieve excellent rich and uniform conditioning of the hair ends/tips on in such a way that rheological parameters such as viscosity are preferred to be high. However, such compositions would not be able to dispense from package, esp. applicators and would not uniformly spread through the hair where the hair can be heavy and weigh down. Thus, it has heretofore been believed that an adequate viscosity of a conditioning composition is necessary to achieve uniform richness of conditioning as to deliver benefits.
   i. Liquid composition dispensing using hands without an applicator:
      The composition may have a zero shear viscosity value measured at 0.01 Pa of from about 4,500 Pa s to about 50,000 Pa s, from about 5,000 Pa s to about 30,000 Pa s, and in a further embodiment from about 6,000 Pa s to about 20,000 Pa s.
         Such a liquid composition may be thick enough to not drip from hair.
   ii. Liquid Composition Dispensing with an Applicator (Non-Foaming)
      The composition may be shear thinning and has (1) a zero shear viscosity value measured at 0.01 Pa of greater than about 3,500 Pa s to about 50,000 Pa s, in an embodiment from about 4,000 Pa s to about 30,000 Pa s, and in a further embodiment from about 5,000 Pa s to about 20,000 Pa s; (2) a moderate stress viscosity value measured at 1 Pa viscosity of greater than about 1,000 Pa s and less than 15,000 Ps, in an embodiment from about 1,500 Pa s to about 13,500 Pa s, in a further embodiment from about 2,000 Pa s to about 12,000 Pa s; and (3) a high shear rate viscosity value measured at 100 l/s of less than about 800 Pa s, in an embodiment less than about 700 Pa s, in a further embodiment less than about 600 Pa s.
Such a composition may provide shear thinning through the applicator for ease of dispensing. Likewise, such a composition may be thick enough for not dripping from hair.
Foam may provide low viscosity uniform spreading, lathering and coverage on scalp and through the hair, not dripping. Viscosity may need to be low enough for such foaming.
   iii. Liquid Composition Dispensing with a Foam Applicator

Such a composition may have a zero shear viscosity value measured at 0.01 Pa of less than 15,000 Pa s, in an embodiment less than about 10,000 Pa s, in a further embodiment less than about 8,000 Pa s.

In one embodiment of the invention, a lower viscosity for a scalp cleansing composition may result in % increase scalp deposition.

### Applicator

In the shampoo process, cleaning needs are highest at the scalp and root. Also, scalp actives need to be delivered to the scalp to provide the benefit. Therefore, it is most beneficial to maximize the concentration of cleaning agents and scalp actives directly to this area of the scalp. However, typical shampoo protocols are applied to the outside surface of the hair and mixed with water to reduce its viscosity and enable cleansing agents and scalp actives to travel to the scalp. Dispensing directly onto the scalp via a targeted delivery applicator enables deposition of the non diluted cleaning agents and scalp actives directly where the cleaning needs and scalp benefit needs are highest. The applicator is attached or can be attached to a bottle containing the cleansing product. The applicator can consist of a base that holds or extends to a single or plurality of tines. The tines have openings that may be at the tip, the base or at any point between the tip and the base. These openings allows for the product to be distributed from the bottle directly onto the hair and/or scalp.

Alternatively, the applicator can also consist of brush-like bristles attached or extending from a base. In this case product would dispense from the base and the bristles would allow for product distribution via the combing or brushing motion.

Applicators as described above may also be leveraged to enable targeted application of the conditioning product. In this case it may be most beneficial for the openings to be located at the base or between the base and the tips.

Applicator and tine design and materials can also be optimized to enable scalp massage. In this case it would be beneficial for the tine or bristle geometry at the tips to be more rounded similar to the roller ball applicator used for eye creams. It may also be beneficial for materials to be smoother and softer; for example metal or metal-like finishes, “rubbery materials.

In an embodiment of the present invention, a root area may be 5 mm of hair closest to the scalp.

In a further embodiment of the present invention, the root area may be a) 50% of the total length of the hair starting at the end attached to the scalp, In a further embodiment, the root area may be b) the 25 cm closest to the scalp starting from the scalp, in a further embodiment, c) the 15 cm closest to the scalp starting from the scalp, in a further embodiment d) the 5 cm closest to the scalp starting from the scalp, or in a further embodiment wherein the 25, or in an embodiment wherein b, c and d in the crown area where crown area is defined as any hair above the ear line.

Scalp Active Material

The composition comprises a scalp active material, which may be an anti-dandruff active particulate. In an embodiment, the anti-dandruff active is selected from the group consisting of: pyridinethione salts; zinc carbonate; azoles, such as ketoconazole, econazole, and elubiol; selenium sulphide; particulate sulfur; keratolytic agents such as salicylic acid; and mixtures thereof. In an embodiment, the anti-dandruff particulate is a pyridinethione salt. Such anti-dandruff particulate should be physically and chemically compatible with the components of the composition, and should not otherwise unduly impair product stability, aesthetics or performance.

Pyridinethione particulates are suitable particulate anti-dandruff actives for use in composition of the present invention. In an embodiment, the anti-dandruff active is a 1-hydroxy-2-pyridinethione salt and is in particulate form. In an embodiment, the concentration of pyridinethione anti-dandruff particulate ranges from about 0.01% to about 5%, by weight of the composition, or from about 0.1% to about 3%, or from about 0.1% to about 2%. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”; zinc pyrithione), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

In an embodiment, in addition to the anti-dandruff active selected from polyvalent metal salts of pyrithione, the composition further comprises one or more anti-fungal and/or anti-microbial actives. In an embodiment, the anti-microbial active is selected from the group consisting of: coal tar, sulfur, charcoal, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of: itraconazole, ketoconazole, selenium sulphide, coal tar, and mixtures thereof.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the composition, the azole anti-microbial active is included in an amount of from about 0.01% to about 5%, or from about 0.1% to about 3%, or from about 0.3% to about 2%, by total weight of the composition. In an embodiment, the azole anti-microbial active is ketoconazole. In an embodiment, the sole anti-microbial active is ketoconazole.

The present invention may also comprise a combination of anti-microbial actives. In an embodiment, the combination of anti-microbial active is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In an embodiment, the composition comprises an effective amount of a zinc-containing layered material. In an embodiment, the composition comprises from about 0.001% to about 10%, or from about 0.01% to about 7%, or from about 0.1% to about 5% of a zinc-containing layered material, by total weight of the composition.

Zinc-containing layered materials may be those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In an embodiment, the ZLM is selected from the group consisting of: hydrozincite (zinc carbonate hydroxide), basic zinc carbonate, aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the ZLM is a layered double hydroxide conforming to the formula [M²⁺₁₋ₓM³⁺ₓ(OH)₂]^{x+}A^{m-}_{x/m}·nH₂O wherein some or all of the divalent ions (M²⁺) are zinc ions (Crepaldi, E L, Pava, P C, Tronto, J, Valim, J B J. Colloid Interfac. Sci. 2002, 248, 429-42).

Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺Aⁿ⁻_{(1=3y)/n}·nH₂O where the two metal ions (M²⁺) may be the same or different. If they are the same and represented by zinc, the formula simplifies to [Zn₁₊ₓ(OH)₂]^{2x+}2x A⁻·nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replace the monovalent anion. These materials can also be formed in situ in a composition or in or during a production process.

In an embodiment, the composition comprises basic zinc carbonate. Commercially available sources of basic zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA). Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The on-scalp deposition of the scalp active material, and in an embodiment, an anti-dandruff active, is at least about 1 microgram/cm². The on-scalp deposition of the scalp active material, such as an anti-dandruff active is important in view of ensuring that the anti-dandruff active reaches the scalp where it is able to perform its function. In an embodiment, the deposition of the scalp active material, such as an anti-dandruff active on the scalp is at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the scalp active material, such as anti-dandruff active is measured by having the hair of individuals washed with a composition comprising an anti-dandruff active, for example a composition pursuant to the present invention, by trained a cosmetician according to a conventional washing protocol. The hair is then parted on an area of the scalp to allow an open-ended glass cylinder to be held on the surface while an aliquot of an extraction solution is added and agitated prior to recovery and analytical determination of anti-dandruff active content by conventional methodology, such as HPLC.

### Scalp Health Actives

In an embodiment of the present invention, the present invention may further comprise one or more additional scalp health actives. This group of materials is varied and provides a wide range of benefits including moisturization, barrier improvement, vitamins, lipid soluble vitamins, anti-dandruff, anti-fungal, anti-microbial, anti-oxidant, anti-itch, and sensates. Such skin health actives include but are not limited to: vitamin E and F, salicylic acid, glycols, glycolic acid, PCA, PEGs, erythritol, glycerin, lactates, niacinamide, hyaluronates, allantoin and other ureas, betaines, sorbitol, glutamates, xylitols, menthol, menthyl lactate, iso cyclomone, benzyl alcohol, perfumes, brighteners, enzymes, sensates, attractants, dyes, pigments, bleaches, aloe, a compound comprising the following structure:

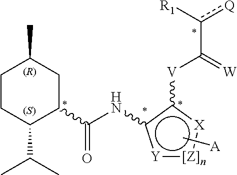

R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓ where x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≧1 and Z is selected from aliphatic CH₂, aromatic CH, or heteroatom;
A=lower alkoxy, lower alkylthio, aryl, substituted aryl or fused aryl; and stereochemistry is variable at the positions marked*;
and
natural extracts/oils including peppermint, spearmint, argan, jojoba, aloe. and mixtures thereof.

Deposition Polymer

The shampoo composition also comprises a cationic deposition polymer. These cationic deposition polymers can include at least one of (a) a cationic guar polymer, (b) a cationic non-guar galactomannan polymer, (c) a cationic tapioca polymer, (d) a cationic copolymer of acrylamide monomers and cationic monomers, and/or (e) a synthetic, non-crosslinked, cationic polymer, which may or may not form lyotropic liquid crystals upon combination with the detersive surfactant (f) a cationic cellulose polymer. Additionally, the cationic deposition polymer can be a mixture of deposition polymers.

(1) Cationic Guar Polymers

According to an embodiment of the present invention, the shampoo composition comprises a cationic guar polymer, which is a cationically substituted galactomannan (guar) gum derivatives. Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums are obtained by reaction between the hydroxyl groups of the polygalactomannan and reactive quaternary ammonium compounds. The degree of substitution of the cationic groups onto the guar structure must be sufficient to provide the requisite cationic charge density described above.

According to one embodiment, the cationic guar polymer has a weight average M.Wt. of less than about 2.5 million g/mol, and has a charge density of from about 0.05 meq/g to about 2.5 meq/g. In an embodiment, the cationic guar polymer has a weight average M.Wt. of less than 1.5 million g/mol, or from about 150 thousand to about 1.5 million g/mol, or from about 200 thousand to about 1.5 million g/mol, or from about 300 thousand to about 1.5 million g/mol, or from about 700,000 thousand to about 1.5 million g/mol. In one embodiment, the cationic guar polymer has a charge density of from about 0.2 to about 2.2 meq/g, or from about 0.3 to about 2.0 meq/g, or from about 0.4 to about 1.8 meq/g; or from about 0.5 meq/g to about 1.7 meq/g.

According to one embodiment, the cationic guar polymer has a weight average M.Wt. of less than about 1 million g/mol, and has a charge density of from about 0.1 meq/g to about 2.5 meq/g. In an embodiment, the cationic guar polymer has a weight average M.Wt. of less than 900 thousand g/mol, or from about 150 thousand to about 800 thousand g/mol, or from about 200 thousand to about 700 thousand g/mol, or from about 300 thousand to about 700 thousand g/mol, or from about 400 thousand to about 600 thousand g/mol. From about 150 thousand to about 800 thousand g/mol, or from about 200 thousand to about 700 thousand g/mol, or from about 300 thousand to about 700 thousand g/mol, or from about 400 thousand to about 600 thousand g/mol. In one embodiment, the cationic guar polymer has a charge density of from about 0.2 to about 2.2 meq/g, or from about 0.3 to about 2.0 meq/g, or from about 0.4 to about 1.8 meq/g; or from about 0.5 meq/g to about 1.5 meq/g.

In an embodiment, the composition comprises from about 0.01% to less than about 0.7%, or from about 0.04% to about 0.55%, or from about 0.08% to about 0.5%, or from about 0.16% to about 0.5%, or from about 0.2% to about 0.5%, or from about 0.3% to about 0.5%, or from about 0.4% to about 0.5%, of cationic guar polymer (a), by total weight of the composition.

The cationic guar polymer may be formed from quaternary ammonium compounds. In an embodiment, the quaternary ammonium compounds for forming the cationic guar polymer conform to the general formula 1:

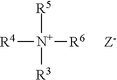

wherein where R³, R⁴ and R⁵ are methyl or ethyl groups; R⁶ is either an epoxyalkyl group of the general formula 2:

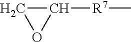

or R⁶ is a halohydrin group of the general formula 3:

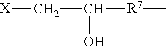

wherein R⁷ is a C₁ to C₃ alkylene; X is chlorine or bromine, and Z is an anion such as Cl—, Br—, I—or HSO₄—.

In an embodiment, the cationic guar polymer conforms to the general formula 4:

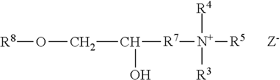

wherein R⁸ is guar gum; and wherein R⁴, R⁵, R⁶ and R⁷ are as defined above; and wherein Z is a halogen. In an embodiment, the cationic guar polymer conforms to Formula 5:

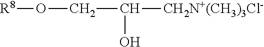

Suitable cationic guar polymers include cationic guar gum derivatives, such as guar hydroxypropyltrimonium chloride. In an embodiment, the cationic guar polymer is a guar hydroxypropyltrimonium chloride. Specific examples of guar hydroxypropyltrimonium chlorides include the Jaguar® series commercially available from Rhone-Poulenc Incorporated, for example Jaguar® C-500, commercially available from Rhodia. Jaguar® C-500 has a charge density of 0.8 meq/g and a M.Wt. of 500,000 g/mole. Jaguar® C-17, which has a cationic charge density of about 0.6 meq/g and a M.Wt. of about 2.2 million g/mol and is available from Rhodia Company. Jaguar® C 13S which has a M.Wt. of 2.2 million g/mol and a cationic charge density of about 0.8 meq/g (available from Rhodia Company). Other suitable guar hydroxypropyltrimonium chloride are: guar hydroxypropyltrimonium chloride which has a charge density of about 1.1 meq/g and a M.Wt. of about 500,000 g/mole is available from ASI, a charge density of about 1.5 meq/g and a M.Wt. of about 500,000 g/mole is available from ASI.

Other suitable guar hydroxypropyltrimonium chloride are: Hi-Care 1000, which has a charge density of about 0.7 meq/g and a M.Wt. of about 600,000 g/mole and is available from Rhodia; N-Hance 3269 and N-Hance 3270, which has a charge density of about 0.7 meq/g and a

M.Wt. of about 425,000 g/mole and is available from ASI; N-Hance 3196, which has a charge density of about 0.8 and a M. Wt. Of about 1,100,000 g/mole and is available from ASI. AquaCat CG518 has a charge density of about 0.9 meq/g and a M.Wt. of about 50,000 g/mole and is available from ASI. BF-13, which is a borate (boron) free guar of charge density of about 1.1 meq/g and M. W.t of about 800,000 and BF-17, which is a borate (boron) free guar of charge density of about 1.7 meq/g and M. W.t of about 800,000 both available from ASI.

(2) Cationic Non-Guar Galactomannan Polymers

The shampoo compositions of the present invention comprise a galactomannan polymer derivative having a mannose to galactose ratio of greater than 2:1 on a monomer to monomer basis, the galactomannan polymer derivative selected from the group consisting of a cationic galactomannan polymer derivative. As used herein, the term “cationic galactomannan” refers to a galactomannan polymer to which a cationic group is added. Non Guar Galactomannan polymer derivatives of the present invention have a ratio of mannose to galactose of greater than 2:1 on a monomer to monomer basis. Suitable ratios of mannose to galactose can be greater than about 3:1, and the ratio of mannose to galactose can be greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose).

In one embodiment of the invention, the non-guar galactomannan polymer derivatives have a M. Wt. from about 1,000 to about 10,000,000, and/or form about 5,000 to about 3,000,000.

The shampoo compositions of the present invention include galactomannan polymer derivatives which have a cationic charge density from about 0.5 meq/g to about 7 meq/g. In one embodiment of the present invention, the galactomannan polymer derivatives have a cationic charge density from about 1 meq/g to about 5 meq/g. The degree of substitution of the cationic groups onto the galactomannan structure should be sufficient to provide the requisite cationic charge density.

In one embodiment of the invention the cationic non-guar galactomannan has a ratio of mannose to galactose is greater than about 4:1, a M.Wt. of about 100,000 to about 500,000, and/or from about 150,000 to about 400,000 and a cationic charge density from about 1 meq/g to about 5 meq/g, and/or from 2 meq/g to about 4 meq/g and is a derived from a cassia plant.

The shampoo compositions of the present invention comprise at least about 0.05% of a galactomannan polymer derivative by weight of the composition. In one embodiment of the present invention, the shampoo compositions comprise from about 0.05% to about 2%, by weight of the composition, of a galactomannan polymer derivative.

(3) Cationically Modified Starch Polymer

The shampoo compositions of the present invention comprise water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight.

The shampoo compositions of the present invention comprise cationically modified starch polymers at a range of about 0.01% to about 10%, and/or from about 0.05% to about 5%, by weight of the composition.

The cationically modified starch polymers disclosed herein have a percent of bound nitrogen of from about 0.5% to about 4%.

The cationically modified starch polymers for use in the shampoo compositions of the present invention have a molecular weight from about 850,000 to about 15,000,000 and/or from about 900,000 to about 5,000,000.

The shampoo compositions of the present invention include cationically modified starch polymers which have a charge density of from about 0.2 meq/g to about 5 meq/g, and/or from about 0.2 meq/g to about 2 meq/g. The chemical modification to obtain such a charge density includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, 0. B., Ed., CRC Press, Inc., Boca Raton, Fla. 1986, pp 113-125. The cationic groups may be added to the starch prior to degradation to a smaller molecular weight or the cationic groups may be added after such modification.

The source of starch before chemical modification can be chosen from a variety of sources such as tubers, legumes, cereal, and grains. Non-limiting examples of this source starch may include corn starch, wheat starch, rice starch, waxy corn starch, oat starch, cassaya starch, waxy barley, waxy rice starch, glutenous rice starch, sweet rice starch, amioca, potato starch, tapioca starch, oat starch, sago starch, sweet rice, or mixtures thereof.

(4) Cationic Copolymer of an Acrylamide Monomer and a Cationic Monomer

According to an embodiment of the present invention, the shampoo composition comprises a cationic copolymer of an acrylamide monomer and a cationic monomer, wherein the copolymer has a charge density of from about 1.0 meq/g to about 3.0 meq/g. In an embodiment, the cationic copolymer is a synthetic cationic copolymer of acrylamide monomers and cationic monomers.

In an embodiment, the cationic copolymer comprises:
(i) an acrylamide monomer of the following Formula AM:

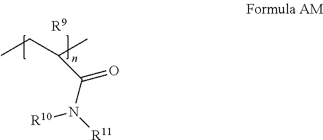

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and
(ii) a cationic monomer conforming to Formula CM:

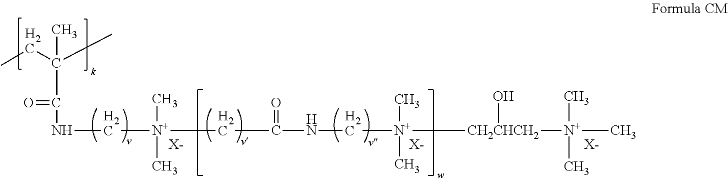

where k=1, each of v, v′, and v″ is independently an integer of from 1 to 6, w is zero or an integer of from 1 to 10, and X⁻ is an anion.

In an embodiment, cationic monomer conforming to Formula CM and where k=1, v=3 and w=0, z=1 and X⁻ is Cl⁻ to form the following structure:

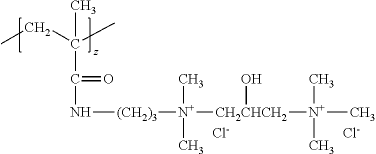

The above structure may be referred to as diquat. In another embodiment, the cationic monomer conforms to Formula CM and wherein v and v″ are each 3, v′=1, w=1, y=1 and X⁻ is Cl⁻, such as:

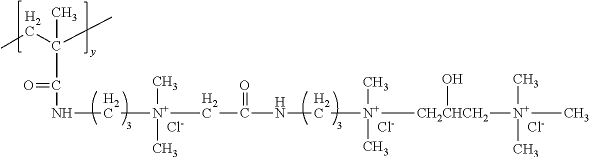

The above structure may be referred to as triquat.

In an embodiment, the acrylamide monomer is either acrylamide or methacrylamide.

In an embodiment, the cationic copolymer (b) is AM:TRIQUAT which is a copolymer of acrylamide and 1,3-Propanediaminium,N-[2-[[[dimethyl[3-[(2-methyl-1-oxo-2-propenyl)amino]propyl]ammonio]acetyl] amino]ethyl]2-hydroxy-N,N,N′,N′,N′-pentamethyl-, trichloride. AM:TRIQUAT is also known as polyquaternium 76 (PQ76). AM:TRIQUAT may have a charge density of 1.6 meq/g and a M.Wt. of 1.1 million g/mol.

In an alternative embodiment, the cationic copolymer is of an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

In an embodiment, the cationic copolymer comprises a cationic monomer selected from the group consisting of: cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

In an embodiment, the cationic copolymer is water-soluble. In an embodiment, the cationic copolymer is formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters may be cationized esters of the (meth)acrylic acid containing a quaternized N atom. In an embodiment, cationized esters of the (meth)acrylic acid containing a quaternized N atom are quaternized dialkylaminoalkyl (meth)acrylates with C1 to C3 in the alkyl and alkylene groups. In an embodiment, the cationized esters of the (meth)acrylic acid containing a quaternized N atom are selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. In an embodiment, the cationized esters of the (meth)acrylic acid containing a quaternized N atom is dimethylaminoethyl acrylate, which is quaternized with an alkyl halide, or with methyl chloride or benzyl chloride or dimethyl sulfate (ADAME-Quat). In an embodiment, the cationic monomer when based on (meth)acrylamides are quaternized dialkylaminoalkyl(meth)acrylamides with C1 to C3 in the alkyl and alkylene groups, or dimethylaminopropylacrylamide, which is quaternized with an alkyl halide, or methyl chloride or benzyl chloride or dimethyl sulfate.

In an embodiment, the cationic monomer based on a (meth)acrylamide is a quaternized dialkylaminoalkyl(meth)acrylamide with C1 to C3 in the alkyl and alkylene groups. In an embodiment, the cationic monomer based on a (meth)acrylamide is dimethylaminopropylacrylamide, which is quaternized with an alkyl halide, especially methyl chloride or benzyl chloride or dimethyl sulfate.

In an embodiment, the cationic monomer is a hydrolysis-stable cationic monomer. Hydrolysis-stable cationic monomers can be, in addition to a dialkylaminoalkyl(meth)acrylamide, all monomers that can be regarded as stable to the OECD hydrolysis test. In an embodiment, the cationic monomer is hydrolysis-stable and the hydrolysis-stable cationic monomer is selected from the group consisting of: diallyldimethylammonium chloride and water-soluble, cationic styrene derivatives.

In an embodiment, the cationic copolymer is a terpolymer of acrylamide, 2-dimethylammoniumethyl (meth)acrylate quaternized with methyl chloride (ADAME-Q) and 3-dimethylammoniumpropyl(meth)acrylamide quaternized with methyl chloride (DIMAPA-Q). In an embodiment, the cationic copolymer is formed from acrylamide and acrylamidopropyltrimethylammonium chloride, wherein the acrylamidopropyltrimethylammonium chloride has a charge density of from about 1.0 meq/g to about 3.0 meq/g.

In an embodiment, the cationic copolymer has a charge density of from about 1.1 meq/g to about 2.5 meq/g, or from about 1.1 meq/g to about 2.3 meq/g, or from about 1.2 meq/g to about 2.2 meq/g, or from about 1.2 meq/g to about 2.1 meq/g, or from about 1.3 meq/g to about 2.0 meq/g, or from about 1.3 meq/g to about 1.9 meq/g.

In an embodiment, the cationic copolymer has a M.Wt. from about 100 thousand g/mol to about 2 million g/mol, or from about 300 thousand g/mol to about 1.8 million g/mol, or from about 500 thousand g/mol to about 1.6 million g/mol, or from about 700 thousand g/mol to about 1.4 million g/mol, or from about 900 thousand g/mol to about 1.2 million g/mol.

In an embodiment, the cationic copolymer is a trimethylammoniopropylmethacrylamide chloride-N-Acrylamide copolymer, which is also known as AM:MAPTAC. AM:MAPTAC may have a charge density of about 1.3 meq/g and a M.Wt. of about 1.1 million g/mol. In an embodiment, the cationic copolymer is AM:ATPAC. AM:ATPAC may have a charge density of about 1.8 meq/g and a M.Wt. of about 1.1 million g/mol.

(5) Cationic Synthetic Polymer

According to an embodiment of the present invention, the shampoo composition comprises a cationic synthetic polymer that may be formed from

i) one or more cationic monomer units, and optionally

ii) one or more monomer units bearing a negative charge, and/or

iii) a nonionic monomer,

wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers

In one embodiment, the cationic polymers are water soluble or dispersible, non-crosslinked, and synthetic cationic polymers having the following structure:

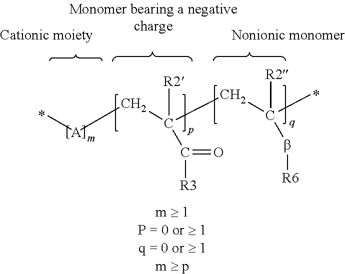

where A, may be one or more of the following cationic moieties:

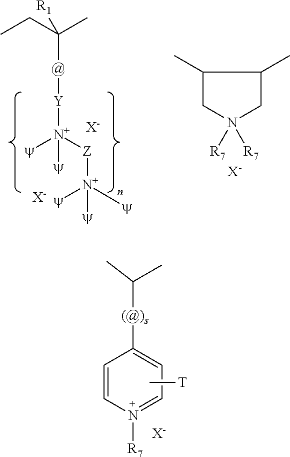

where @=amido, alkylamido, ester, ether, alkyl or alkylaryl;
where Y=C1-C22 alkyl, alkoxy, alkylidene, alkyl or aryloxy;
where ψ=C1-C22 alkyl, alkyloxy, alkyl aryl or alkyl arylox;
where Z=C1-C22 alkyl, alkyloxy, aryl or aryloxy;
where R1=H, C1-C4 linear or branched alkyl;
where s=0 or 1, n=0 or ≧1;
where T and R7=C1-C22 alkyl; and
where X−=halogen, hydroxide, alkoxide, sulfate or alkylsulfate.

Where the monomer bearing a negative charge is defined by R2′=H, C1-C4 linear or branched alkyl and R3 as:

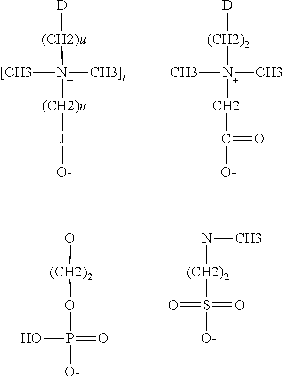

where D=O,N, or S;
where Q=NH₂ or O;
where u=1-6;
where t=0-1; and
where J=oxygenated functional group containing the following elements P, S, C.

Where the nonionic monomer is defined by R2″=H, C1-C4 linear or branched alkyl, R6=linear or branched alkyl, alkyl aryl, aryl oxy, alkyloxy, alkylaryl oxy and β is defined as

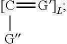

and where G′ and G″ are, independently of one another, O, S or N—H and L=0 or 1.

Examples of cationic monomers include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of cationic monomers include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers include those which comprise a quaternary ammonium group of formula —NR₃⁺, wherein R, which is identical or different, represents a hydrogen atom, an alkyl group comprising 1 to 10 carbon atoms, or a benzyl group, optionally carrying a hydroxyl group, and comprise an anion (counter-ion). Examples of anions are halides such as chlorides, bromides, sulphates, hydrosulphates, alkylsulphates (for example comprising 1 to 6 carbon atoms), phosphates, citrates, formates, and acetates.

Suitable cationic monomers include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride.

Additional suitable cationic monomers include trimethyl ammonium propyl (meth)acrylamido chloride.

Examples of monomers bearing a negative charge include alpha ethylenically unsaturated monomers comprising a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds.

Suitable nonionic monomers include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X—) in association with the synthetic cationic polymers may be any known counterion so long as the polymers remain soluble or dispersible in water, in the shampoo composition, or in a coacervate phase of the shampoo composition, and so long as the counterions are physically and chemically compatible with the essential components of the shampoo composition or do not otherwise unduly impair product performance, stability or aesthetics. Non limiting examples of such counterions include halides (e.g., chlorine, fluorine, bromine, iodine), sulfate and methylsulfate.

The concentration of the cationic polymers ranges about 0.025% to about 5%, from about 0.1% to about 3%, and/or from about 0.2% to about 1%, by weight of the shampoo composition.

(6) Cationic Cellulose Polymers

Suitable cationic cellulose polymers are salts of hydroxyethyl cellulose reacted with trimethyl ammonium substituted epoxide, referred to in the industry (CTFA) as Polyquaternium 10 and available from Dow/Amerchol Corp. (Edison, N.J., USA) in their Polymer LR, JR, and KG series of polymers. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H.

In an embodiment, the shampoo composition comprises a plurality of cationic conditioning polymers. According to one embodiment, where two cationic conditioning polymers are present, the weight ratio of a first cationic conditioning polymer to a second cationic conditioning polymer is from about 1000:1 to about 2:1. In an embodiment, the weight ratio of the first cationic conditioning polymer to the second cationic conditioning polymer is from about 1000:1 to about 4:1. In an embodiment, weight ratio of the first cationic conditioning polymer to the second cationic conditioning polymer is from about 800:1 to about 4:1, or from about 500:1 to about 4:1, or from about 100:1 to about 5:1, or from about 100:1 to about 6:1, or from about 50:1 to about 6.5:1, or from about 50:1 to about 7:1, or from about 50:1 to about 8.3:1, or from about 50:1 to about 16.7:1

Carrier

The composition comprises a cosmetically acceptable carrier. In an embodiment, the carrier is an aqueous carrier. The amount and chemistry of the carrier is selected according to the compatibility with other components and other desired characteristic of the product. In an embodiment, the carrier is selected from the group consisting of: water and water solutions of lower alkyl alcohols. In an embodiment, the carrier is a lower alkyl alcohol, wherein the monohydric alcohol has 1 to 6 carbons. In an embodiment, the carrier is ethanol and/or isopropanol. In an embodiment, the cosmetically acceptable carrier is a cosmetically acceptable aqueous carrier and is present at a level of from about 20% to about 95%, or from about 60% to about 85%.

Surfactant

The composition comprises a surfactant. The surfactant is included to provide cleaning performance to the composition. In an embodiment, the surfactant is selected from the group consisting of: anionic surfactants, amphoteric surfactants, zwitterionic surfactants, cationic surfactants, non-ionic surfactants, and mixtures thereof. In an embodiment, the surfactant is an anionic surfactant. In an embodiment, the composition comprises from about 5% to about 50%, or from about 8% to about 30%, or from about 10% to about 25% of a surfactant, by total weight of the composition.

The composition may comprise a detersive surfactant system. The detersive surfactant system may comprise at least one anionic surfactant, and optionally a co-surfactant selected from the group consisting of: an amphoteric surfactant, a zwitterionic surfactant, a cationic surfactant, a nonionic surfactant, or a mixture thereof. The concentration of the detersive surfactant system in the composition should be sufficient to provide the desired cleaning and lather performance. In an embodiment, the composition comprises from about 5% to about 50%, or from about 8% to about 30%, or from about 10% to about 25% of detersive surfactant system, by total weight of the composition.

In considering the performance characteristics, such as coacervate formation, wet conditioning performance, dry conditioning performance, and conditioning agent deposition on hair, it is desirable to optimize the levels and types of surfactants in order to maximize the performance potential of polymer systems. In one embodiment, the detersive surfactant system for use in the composition comprises an anionic surfactant with an ethoxylate level and an anion level, wherein the ethoxylate level is from about 1 to about 10, and wherein the anion level is from about 1 to about 10. The combination of such an anionic surfactant with the cationic copolymer and cationic guar polymer provides enhanced deposition of conditioning agents to hair and/or skin without reducing cleansing or lathering performance. An optimal ethoxylate level is calculated based on the stoichiometry of the surfactant structure, which in turn is based on a particular M.Wt. of the surfactant where the number of moles of ethoxylation is known. Likewise, given a specific M.Wt. of a surfactant and an anionization reaction completion measurement, the anion level can be calculated.

In an embodiment, the detersive surfactant system comprises at least one anionic surfactant comprising an anion selected from the group consisting of sulfates, sulfonates, sulfosuccinates, isethionates, carboxylates, phosphates, and phosphonates. In an embodiment, the anion is a sulfate.

In an embodiment, the anionic surfactant is an alkyl sulfate or an alkyl ether sulfate. These materials have the respective formulae R⁹OSO₃M and R⁹O(C₂H₄O)ₓSO₃M, wherein R⁹ is alkyl or alkenyl of from about 8 to about 18 carbon atoms, x is an integer having a value of from about 1 to about 10, and M is a cation such as ammonium, an alkanolamine such as triethanolamine, a monovalent metal cation such as sodium and potassium, or a polyvalent metal cation such as magnesium and calcium. Solubility of the surfactant will depend upon the particular anionic surfactants and cations chosen. In an embodiment, R⁹ has from about 8 to about 18 carbon atoms, or from about 10 to about 16 carbon atoms, or from about 12 to about 14 carbon atoms, in both the alkyl sulfates and alkyl ether sulfates. The alkyl ether sulfates are typically made as condensation products of ethylene oxide and monohydric alcohols having from about 8 to about 24 carbon atoms. The alcohols can be synthetic or they can be derived from fats, e.g., coconut oil, palm kernel oil, tallow. In an embodiment, the alcohols are lauryl alcohol and straight chain alcohols derived from coconut oil or palm kernel oil. Such alcohols are reacted with from about 0 to about 10, or from about 2 to about 5, or about 3, molar proportions of ethylene oxide, and the resulting mixture of molecular species having, for example, an average of 3 moles of ethylene oxide per mole of alcohol is sulfated and neutralized. In an embodiment, the alkyl ether sulphate is selected from the group consisting of: sodium and ammonium salts of coconut alkyl triethylene glycol ether sulfate, tallow alkyl triethylene glycol ether sulfate, tallow alkyl hexa-oxyethylene sulphate, and mixtures thereof. In an embodiment, the alkyl ether sulfate comprises a mixture of individual compounds, wherein the compounds in the mixture have an average alkyl chain length of from about 10 to about 16 carbon atoms and an average degree of ethoxylation of from about 1 to about 4 moles of ethylene oxide. Such a mixture also comprises from about 0% to about 20% C₁₂₋₁₃ compounds; from about 60% to about 100% of C₁₄₋₁₅₋₁₆ compounds; from about 0% to about 20% by weight of _{C17-18-19} compounds; from about 3% to about 30% by weight of compounds having a degree of ethoxylation of 0; from about 45% to about 90% by weight of compounds having a degree of ethoxylation from about 1 to about 4; from about 10% to about 25% by weight of compounds having a degree of ethoxylation from about 4 to about 8; and from about 0.1% to about 15% by weight of compounds having a degree of ethoxylation greater than about 8.

In an embodiment, the anionic surfactant is selected from the group consisting of: ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, and mixtures thereof. In addition to the sulfates, isethionates, sulfonates, sulfosuccinates described above, other potential anions for the anionic surfactant include phosphonates, phosphates, and carboxylates.

In an embodiment, the composition of the present invention can also include anionic surfactants selected from the group consisting of:

a) R₁O(CH₂CHR₃O)_{y}SO₃M;

b) CH₃(CH₂)_{z}CHR₂CH₂O(CH₂CHR₃O)_{y}SO₃M; and

c) mixtures thereof,

where R₁ represents CH₃ (CH₂)₁₀, R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

The composition and/or the detersive surfactant system may comprise a co-surfactant selected from the group consisting of: amphoteric surfactants, zwitterionic surfactants, cationic surfactants, non-ionic surfactants, and mixtures thereof. The concentration of such co-surfactants may be from about 0.5% to about 20%, or from about 1% to about 10%, by total weight of the composition. In an embodiment, the composition comprises a co-surfactant selected from the group consisting of: amphoteric surfactants, zwitterionic surfactants, and mixtures thereof. Non limiting examples of suitable zwitterionic or amphoteric surfactants are described in U.S. Pat. No. 5,104,646 (Bolich Jr. et al.), U.S. Pat. No. 5,106,609 (Bolich Jr. et al.).

Amphoteric surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. In an embodiment, the amphoteric surfactant is selected from the group consisting of: sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethy-7 carboxyamphodiacetate, lauraminopropionic acid, lauroamphodipropionic acid, lauryl aminopropylglycine, lauryl diethylenediaminoglycine, and mixtures thereof.

In one embodiment, the amphoteric surfactant is a surfactant according to the following structure:

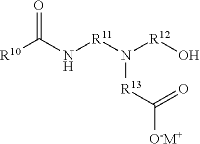

wherein R¹⁰ is a C-linked monovalent substituent selected from the group consisting of: substituted alkyl systems comprising 9 to 15 carbon atoms, unsubstituted alkyl systems comprising 9 to 15 carbon atoms, straight alkyl systems comprising 9 to 15 carbon atoms, branched alkyl systems comprising 9 to 15 carbon atoms, and unsaturated alkyl systems comprising 9 to 15 carbon atoms; and wherein R¹¹, R¹², and R¹³ are each independently selected from the group consisting of: C-linked divalent straight alkyl systems comprising 1 to 3 carbon atoms, and C-linked divalent branched alkyl systems comprising 1 to 3 carbon atoms; and wherein M⁺ is a monovalent counterion selected from the group consisting of sodium, ammonium and protonated triethanolamine. In an embodiment, the amphoteric surfactant is selected from the group consisting of: sodium cocoamphoacetate, sodium cocoamphodiacetate, sodium lauroamphoacetate, sodium lauroamphodiacetate, ammonium lauroamphoacetate, ammonium cocoamphoacetate, triethanolamine lauroamphoacetate, triethanolamine cocoamphoacetate, and mixtures thereof.

In an embodiment, the composition comprises a zwitterionic surfactant, wherein the zwitterionic surfactant is a derivative of an aliphatic quaternary ammonium, phosphonium, and sulfonium compound, in which the aliphatic radicals are straight or branched chain, and wherein one of the aliphatic substituents contains from about 8 to about 18 carbon atoms and one contains an anionic group such as carboxy, sulfonate, sulfate, phosphate or phosphonate. In an embodiment, the zwitterionic surfactant is selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof. In an embodiment, the zwitterionic surfactant is selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

In an embodiment, the co-surfactant is selected from the group consisting of: zwitterionic surfactants, amphoteric surfactants, non-ionic surfactants, and mixtures thereof. In an embodiment, the surfactant is an anionic surfactant and the composition further comprises a co-surfactant, wherein the co-surfactant is selected from the group consisting of: zwitterionic surfactants, amphoteric surfactants, non-ionic surfactants, and mixtures thereof. In an embodiment, the co-surfactant is a non-ionic surfactant selected from the group consisting of: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, and mixtures thereof. In an embodiment, the co-surfactant is a zwitterionic surfactant, wherein the zwitterionic surfactant is selected from the group consisting of: lauryl hydroxysultaine, cocamidopropyl hydroxysultaine, coco-betaine, coco-hydroxysultaine, coco-sultaine, lauryl betaine, lauryl sultaine, and mixtures thereof.

In an embodiment of the present invention, the conditioning shampoo may contain less than 4% detersive surfactants which provides a cleansing benefit.

Silicones

The conditioning agent of the compositions of the present invention can be a silicone conditioning agent. The silicone conditioning agent may comprise volatile silicone, non-volatile silicone, or combinations thereof. The concentration of the silicone conditioning agent typically ranges from about 0.01% to about 10%, by weight of the composition, from about 0.1% to about 8%, from about 0.1% to about 5%, and/or from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609, which descriptions are incorporated herein by reference. The silicone conditioning agents for use in the compositions of the present invention can have a viscosity, as measured at 25° C., from about 20 to about 2,000,000 centistokes (“csk”), from about 1,000 to about 1,800,000 csk, from about 50,000 to about 1,500,000 csk, and/or from about 100,000 to about 1,500,000 csk.

The dispersed silicone conditioning agent particles typically have a volume average particle diameter ranging from about 0.01 micrometer to about 50 micrometer. For small particle application to hair, the volume average particle diameters typically range from about 0.01 micrometer to about 4 micrometer, from about 0.01 micrometer to about 2 micrometer, from about 0.01 micrometer to about 0.5 micrometer. For larger particle application to hair, the volume average particle diameters typically range from about 5 micrometer to about 125 micrometer, from about 10 micrometer to about 90 micrometer, from about 15 micrometer to about 70 micrometer, and/or from about 20 micrometer to about 50 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

Silicone emulsions suitable for use in the embodiments of the present invention include, but are not limited to, emulsions of insoluble polysiloxanes prepared in accordance with the descriptions provided in U.S. Pat. No. 4,476,282 and U.S. Patent Application Publication No. 2007/0276087. Accordingly, suitable insoluble polysiloxanes include polysiloxanes such as alpha, omega hydroxy-terminated polysiloxanes or alpha, omega alkoxy-terminated polysiloxanes having a molecular weight within the range from about 50,000 to about 500,000 g/mol. The insoluble polysiloxane can have an average molecular weight within the range from about 50,000 to about 500,000 g/mol. For example, the insoluble polysiloxane may have an average molecular weight within the range from about 60,000 to about 400,000; from about 75,000 to about 300,000; from about 100,000 to about 200,000; or the average molecular weight may be about 150,000 g/mol. The insoluble polysiloxane can have an average particle size within the range from about 30 nm to about 10 micron. The average particle size may be within the range from about 40 nm to about 5 micron, from about 50 nm to about 1 micron, from about 75 nm to about 500 nm, or about 100 nm, for example.

The average molecular weight of the insoluble polysiloxane, the viscosity of the silicone emulsion, and the size of the particle comprising the insoluble polysiloxane are determined by methods commonly used by those skilled in the art, such as the methods disclosed in Smith, A. L. The Analytical Chemistry of Silicones, John Wiley & Sons, Inc.: New York, 1991. For example, the viscosity of the silicone emulsion can be measured at 30° C. with a Brookfield viscosimeter with spindle 6 at 2.5 rpm. The silicone emulsion may further include an additional emulsifier together with the anionic surfactant,

Other classes of silicones suitable for use in compositions of the present invention include but are not limited to: i) silicone fluids, including but not limited to, silicone oils, which are flowable materials having viscosity less than about 1,000,000 csk as measured at 25° C.; ii) aminosilicones, which contain at least one primary, secondary or tertiary amine; iii) cationic silicones, which contain at least one quaternary ammonium functional group; iv) silicone gums; which include materials having viscosity greater or equal to 1,000,000 csk as measured at 25° C.; v) silicone resins, which include highly cross-linked polymeric siloxane systems; vi) high refractive index silicones, having refractive index of at least 1.46, and vii) mixtures thereof.

Organic Conditioning Materials

The conditioning agent of the shampoo compositions of the present invention may also comprise at least one organic conditioning material such as oil or wax, either alone or in combination with other conditioning agents, such as the silicones described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

Emusifiers

A variety of anionic and nonionic emulsifiers can be used in the shampoo composition of the present invention. The anionic and nonionic emulsifiers can be either monomeric or polymeric in nature. Monomeric examples include, by way of illustrating and not limitation, alkyl ethoxylates, alkyl sulfates, soaps, and fatty esters and their derivatives. Polymeric examples include, by way of illustrating and not limitation, polyacrylates, polyethylene glycols, and block copolymers and their derivatives. Naturally occurring emulsifiers such as lanolins, lecithin and lignin and their derivatives are also non-limiting examples of useful emulsifiers.

Chelating Agents

The shampoo composition can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term include alkali metal, alkaline earth, ammonium, substituted ammonium (i.e. monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. No. 5,747,440.

Levels of the EDDS chelant in the shampoo compositions can be as low as about 0.01 wt % or even as high as about 10 wt %, but above the higher level (i.e., 10 wt %) formulation and/or human safety concerns may arise. In an embodiment, the level of the EDDS chelant may be at least about 0.05 wt %, at least about 0.1 wt %, at least about 0.25 wt %, at least about 0.5 wt %, at least about 1 wt %, or at least about 2 wt % by weight of the shampoo composition. Levels above about 4 wt % can be used but may not result in additional benefit.

Gel Network

The shampoo composition may also comprise fatty alcohol gel networks. These gel networks are formed by combining fatty alcohols and surfactants in the ratio of from about 1:1 to about 40:1, from about 2:1 to about 20:1, and/or from about 3:1 to about 10:1. The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. The gel network contributes a stabilizing benefit to cosmetic creams and hair conditioners. In addition, they deliver conditioned feel benefits for hair conditioners.

The fatty alcohol can be included in the fatty alcohol gel network at a level by weight of from about 0.05 wt % to about 14 wt %. For example, the fatty alcohol may be present in an amount ranging from about 1 wt % to about 10 wt %, and/or from about 6 wt % to about 8 wt %.

The fatty alcohols useful herein include those having from about 10 to about 40 carbon atoms, from about 12 to about 22 carbon atoms, from about 16 to about 22 carbon atoms, and/or about 16 to about 18 carbon atoms. These fatty alcohols can be straight or branched chain alcohols and can be saturated or unsaturated. Nonlimiting examples of fatty alcohols include cetyl alcohol, stearyl alcohol, behenyl alcohol, and mixtures thereof. Mixtures of cetyl and stearyl alcohol in a ratio of from about 20:80 to about 80:20 are suitable.

Gel network preparation: A vessel is charged with water and the water is heated to about 74° C. Cetyl alcohol, stearyl alcohol, and SLES surfactant are added to the heated water. After incorporation, the resulting mixture is passed through a heat exchanger where the mixture is cooled to about 35° C. Upon cooling, the fatty alcohols and surfactant crystallized to form a crystalline gel network. Table 1 provides the components and their respective amounts for an example gel network composition.

TABLE 1
Gel network components

| Ingredient | Wt. % |
|---|---|
| Water | 78.27% |
| Cetyl Alcohol | 4.18% |
| Stearyl Alcohol | 7.52% |
| Sodium laureth-3 sulfate (28% Active) | 10.00% |
| 5-Chloro-2-methyl-4-isothiazolin-3-one, Kathon CG | 0.03% |

In accordance with embodiments of the present invention, the personal care composition may further comprise one or more benefit agents. Exemplary benefit agents include, but are not limited to, particles, colorants, perfume microcapsules, gel networks, and other insoluble skin or hair conditioning agents such as skin silicones, natural oils such as sun flower oil or castor oil. In an embodiment, the benefit agent is selected from the group consisting of: particles; colorants; perfume microcapsules; gel networks; other insoluble skin or hair conditioning agents such as skin silicones, natural oils such as sun flower oil or castor oil; and mixtures thereof.

A. Optional Ingredients

In accordance with embodiments of the present invention, the hair care composition may further comprise one or more optional ingredients, including benefit agents Suitable benefit agents include, but are not limited to conditioning agents, cationic polymers silicone emulsions, gel networks, chelating agents, and, natural oils such as sun flower oil or castor oil. Additional suitable optional ingredients include but are not limited to perfumes, perfume microcapsules, colorants, particles, anti-microbials, foam busters, anti-static agents, rheology modifiers and thickeners, suspension materials and structurants, pH adjusting agents and buffers, preservatives, pearlescent agents, solvents, diluents, anti-oxidants, vitamins and combinations thereof.

Such optional ingredients should be physically and chemically compatible with the components of the composition, and should not otherwise unduly impair product stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

### Product Form

The hair care compositions of the present invention may be presented in typical hair care formulations. They may be in the form of solutions, dispersion, emulsions, powders, talcs, encapsulated, spheres, spongers, solid dosage forms, foams, and other delivery mechanisms. The compositions of the embodiments of the present invention may be hair tonics, leave-on hair products such as treatment, and styling products, rinse-off hair products such as shampoos and personal cleansing products, and treatment products; and any other form that may be applied to hair.

### Aerosol Foam Dispenser

An aerosol foam dispenser may comprise a reservoir for holding a concentrated hair treatment composition. The reservoir may be made out of any suitable material selected from the group consisting of plastic, metal, alloy, laminate, and combinations thereof. In an embodiment, the reservoir may be for one-time use. In an embodiment, the reservoir may be removable from the aerosol foam dispenser. Alternatively, the reservoir may be integrated with the aerosol foam dispenser. In an embodiment, there may be two or more reservoirs.

In an embodiment, the reservoir may be comprised of a material selected from the group consisting of rigid materials, flexible materials, and combinations thereof. The reservoir may be comprised of a rigid material if it does not collapse under external atmospheric pressure when it is subject to an interior partial vacuum.

### EXAMPLES

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified.

Scalp Cleansing Composition and High Conditioning Shampoo Examples

| Ingredient | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|
| Water | q.s. | q.s. | q.s. | q.s. | q.s. | q.s. | q.s. | q.s. | q.s. | q.s. |
| Sodium Laureth Sulfate | 10 | 10 | 6 | 6 | — | — | — | — | — | 8 |
| (SLE3S-28% active)¹ | | | | | | | | | | |
| Sodium Laureth Sulfate | — | — | — | — | 12 | 10.5 | 11.5 | 10.5 | 16 | — |
| (SLE1S-29% active) ² | | | | | | | | | | |
| Sodium Lauryl Sulfate | 6 | 6 | 7 | 7 | — | 1.5 | — | 1.5 | — | 7 |
| (SLS-29% active) ³ | | | | | | | | | | |
| Coco monoethanolamide | 1.6 | 1.6 | — | — | 1.5 | 1 | 1.5 | 1 | — | — |
| (85% active)⁴ | | | | | | | | | | |
| Cocoamdopropyl Betaine | — | — | 1 | 1 | 1.5 | 1 | 1.25 | 1 | 1.7 | 2 |
| (30% active)⁵ | | | | | | | | | | |
| Ethylene Glycol Disterate⁶ | 1.5 | 1.5 | 2.5 | 2.5 | 1.5 | 1.5 | 2.0 | 1.5 | — | 1.5 |
| Polyquaternium 76⁷ | — | — | — | — | — | 0.001 | 0.003 | 0.001 | — | — |
| Polquaternium 10⁸ | — | — | — | — | — | — | — | — | 0.075 | — |
| Polquaternium 10⁹ | | | | | 0.2 | — | — | | | |
| Polyquaternium 6¹⁰ | — | — | — | — | — | — | — | — | 0.075 | — |
| Guar, Hydroxylpropyl | 0.5 | 0.5 | 0.23 | 0.23 | 0.3 | 0.3 | 0.3 | 0.3 | — | 0.15 |
| Trimonium Chloride¹¹ | | | | | | | | | | |
| Guar, Hydroxylpropyl | — | — | — | — | — | — | — | — | 0.325 | 0.15 |
| Trimonium Chloride¹² | | | | | | | | | | |
| 330M silicone 712¹³ | 0.85 | 1.7 | 0.8 | 0.8 | 0.8 | 0.8 | 0.8 | 1.7 | — | 2.7 |
| Silicone microemulsion¹⁴ | — | — | — | — | — | — | — | — | 0.75 | — |
| Trihydroxysteam¹⁵ | — | — | — | — | — | — | — | — | 0.1 | — |
| Zinc Pyrithone¹⁶ | 1.0 | 1.0 | 1.0 | 1.0 | 1.0 | 1.0 | 1.0 | 2 | — | — |
| Zinc Carbonate¹⁷ | 1.6 | 1.6 | 1.6 | 1.6 | 1.6 | 1.6 | 1.6 | 1.6 | — | — |
| Gel Network¹⁸ | — | — | — | — | — | — | — | — | 17.7 | — |
| Magnesium Sulfate | 0.28 | 0.28 | | | | | | | | |
| Cetyl Alcohol | 0.6 | 0.6 | | | | | | | | |
| Menthol¹⁹ | — | 0.5 | | 0.5 | 0.5 | 0.5 | 0.5 | 0.5 | — | — |
| Fragrance | 0.7 | 0.7 | 0.6 | 0.6 | 0.7 | 0.7 | 0.7 | 0.7 | 0.7 | 0.7 |

| | |
|---|---|
| Sodium Chloride, Sodium | Adjust as needed for viscosity of about 5,000-15,000 cps at 2/s shear rate |
| Xylene Sulfonate²⁰ | |
| Preservatives, pH adjusters | Up to 1% |

¹Sodium Laureth (3 molar ethylene oxide) Sulfate at 28% active, supplier: P&G
² Sodium Laureth (1 molar ethylene oxide) sulfate at 29% active, supplier: P&G
³ Sodium Lauryl Sulfate at 29% active, supplier: P&G
⁴Coco monethanolamide at 85% active, supplier: Stephan Co or supplier Evonik
⁵Tegobetaine F-B, 30% active, supplier: Goldschmidt Chemical
⁶Ethylene Glycol Disterate at 100% active, supplier: Goldschmidt Chemical or supplier Evonik
⁷Acrylamide: Triquat cationic polymer, 10% solution, trade name: Mirapol AT from Rhodia,
⁸KG30M cationic cellulose polymer from Amerchol/Dow
⁹JR30M cationic cellulose polymer from Amerchol/Dow
¹⁰PolyDADMAC at 31.5% active, trade name: Mirapol 100S from Rhodia
¹¹Cationic Guar with M. Wt. of ~500,000 and a Charge Density of 0.8 meq/g - Solvay/Rhodia
¹²Cationic Guar with M. Wt. of ~1,300,000 and a Charge Density of 0.8 meq/g - ASI
¹³330M silicone, 100% active, supplier: Momentive (silicone used by P&G to make a 70% active, 30 um emulsion)
¹⁴Belsil 3560 VP silicone microemulsion from Wacker, 60,000 cst internal viscosity of silicone, approx. 125 nm
¹⁵Thixin R from Rheox Inc.
¹⁶ZPT from Arch Chemical
¹⁷Zinc carbonate from the Bruggeman Group
¹⁸Gel Networks; See Composition below. The water is heated to about 74° C. and the Cetyl Alcohol, Stearyl Alcohol, and the SLES Surfactant are added to it. After incorporation, this mixture is passed through a heat exchanger where it is cooled to about 35° C. As a result of this cooling step, the Fatty Alcohols and surfactant crystallized to form a crystalline gel network.
¹⁹Menthol from Symrise
²⁰Sodium Chloride USP (food grade), Sodium Xylene Sulfonate 30% active

### Example 11

| Ingredient | Wt % |
|---|---|
| Water | q.s. |
| Cetyl Alcohol | 3.46 |
| Steary Alcohol | 6.44 |
| Sodium Laureth Sulfate (SLE3S-28% active) | 3.93 |
| Kathon, (Methylchloroisothiazolinone/ | 0.003 |
| methyisothiazolinone) | |

### Example 12

| Ingredient | Wt % |
|---|---|
| Ammonium Laureth Sulfate | 14.1 |
| Ammoniun Lauryl Sulfate | 3.1 |
| Ammonium Sylenesulfonate | 0.45 |
| TWEEN 60 | 3.0 |
| Polyquaternium-10¹ | 0.35 |
| Tricetylmonium Chloride | 0.5 |
| Selenium Sulfide | 1.0 |
| Dimethicone | 0.60 |
| Ethylene Glycol Distearate | 3.0 |
| Cocamide MEA | 3.0 |
| Cetyl Alcohol | 0.42 |
| DMDM Hyantoin | 0.40 |
| Sodium Chloride | 0.20 |
| Stearyl Alcohol | 0.20 |
| Hydroxypropyl Methylcellulose | 0.02 |
| Water | Q.S. |

¹UCARE Polymer LR400, available from Amerchol
²Visasil 330,000 csk, available from General Electric Silicones

### Methodology

Two separate but identical criterion groups are recruited to participate in product usage. Recruiting criteria for both groups are Female, age 18-65, Shampoo 3× or more/week, with Shoulder length or longer hair that has experienced Dandruff, Dry Scalp or Itch in past 4 weeks. The criteria is chosen to provide a subject with hair and scalp needs. Each group is given a a commercial branded anti-dandruff product description specific with their designated group, either Product A or Product B. Product is given to concept acceptors of the product description to use for a period of 4 weeks. Concept acceptors are defined as those who indicate that they would Definitely or Probably buy the product bases on description and price. At 4 weeks each panelists completes a self administered questionnaire about their experience with the product that they receive (either product A or Product B). Panelists conditioner usage is ad lib at their discretion throughout the usage period.

### Results

| | Product A | Product B |
|---|---|---|
| Attribute | Example 3 | Example 2 |
| base size | 68 | 74 |
| Overall Cleaning | 75 | 82a |
| Overall Conditioning | 57 | 66a |
| Overall Health of my hair | 67 | 75a |
| Overall Achieving the look I want | 62 | 70a |
| Lather being able to get to the scalp | 73 | 81A |
| Making my scalp feel cleaner | 74 | 84A |
| Relieving itch | 69 | 79A |
| Providing a soothing feeling to scalp | 70 | 80A |

Scale: 0 (Poor) −100 (Excellent) Rating
CAPITAL letter denotes statistical significance at 95% confidence
lower case letter denotes statistical significance at 90% confidence

This system with targeted application (Product B)(Example 2) delivers not only noticeable but meaningful improvement versus consumers' approach to scalp care today with approximately 2× more consumers that strongly agree the system delivers better symptom relief and scalp health versus their approach today.

| | % Strongly Agree | |
|---|---|---|
| | Product A | Product B |
| Attribute | Example 3 | Example 2 |
| base size | 68 | 74 |
| The Test Product Provided Me with Better Scalp | 24% | 45% A |
| Symptom Relief Compared to | | |
| My Solution Before the Test | | |
| The Test Product Provided Me with Better | 21% | 47% A |
| Overall Scalp Health Compared to My | | |
| Solution Before the Test | | |

Scale: Strongly Agree to Strongly Disagree
CAPITAL letter denotes statistical significance at 95% confidence
lower case letter denotes statistical significance at 90% confidence

### Results Discussion

The synergy of having specifically formulated shampoos that target differently the roots/scalp and the lengths of the hair differently along with a targeted application that deposits the specific formulated shampoo to the root/scalp region significantly increases overall consumer delight.

Importantly, this system approach (Product B)(Example 2) resolves common performance contradictions and trade-offs beyond today's traditional shampoo application driving superior and noticeable improvement for multiple and often times competing area of scalp, hair cleanliness, and hair conditioning and hair health.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the hair care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of delivering a scalp cleansing composition to the scalp and a conditioning shampoo composition to the rest of the hair comprising:
a. Applying a scalp cleansing composition directly onto the scalp using an applicator wherein the scalp cleansing composition comprises a sensate;
b. Applying a conditioning shampoo composition on the length and ends of the hair, where length is defined as the hair starting below the root line at the back of the neck/base of the head and the ends are defined as the 4 cm of the hair furthest away from the scalp;
c. wherein % increase scalp deposition is 10%.

2. A method of delivering a scalp cleansing composition to the scalp and a conditioning shampoo composition to the rest of the hair according to claim 1 further comprising applying a scalp cleansing composition directly onto the scalp and root area of the hair using an applicator, wherein root area is about 5 mm of hair closest to the scalp.

3. A method of delivering a scalp cleansing composition to the scalp and a conditioning shampoo composition according to claim 1 wherein the conditioning shampoo composition is applied on the length and ends of the hair using an applicator, where length is defined as the hair starting below the root line at the back of the neck/base of the head and the ends are defined as the 4 cm of the hair furthest away from the scalp.

4. A method according to claim 1 wherein the scalp cleansing composition is followed by rinsing with water and further application of a rinse-off hair conditioning composition on the length and ends of the hair, where length is defined as the hair starting below the root line at the back of the neck/base of the head and the ends are defined as the 4 cm of the hair furthest away from the scalp.

5. A method according to claim 1 wherein the scalp cleansing composition is delivered via an applicator having a single and/or plurality of outlets extending more than 1 cm from its base.

6. A method according to claim 1 wherein the conditioning shampoo composition is delivered via an applicator having a single and/or plurality of outlets extending more than 1 cm from its base.

7. A method according to claim 1 wherein the sensate is selected from the group consisting of menthol, peppermint, spearmint, a compound comprising the following structure: 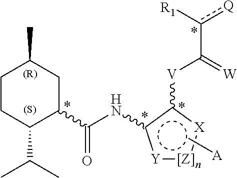
R₁ is selected from H, alkyl, amino alkyl, alkoxy;
Q=H₂, O, —OR₁, —N(R₁)₂, —OPO(OR₁)ₓ, —PO(OR₁)ₓ, —P(OR₁)ₓwhere x=1-2;
V=NR₁, O, —OPO(OR₁)ₓ, —PO(OR₁)ₓ, —P(OR₁)ₓ where x=1-2;
W=H₂, O;
X, Y=independently selected from H, aryl, naphthyl for n=0;
X, Y=aliphatic CH₂ or aromatic CH for n≧1 and Z is selected from aliphatic CH₂, aromatic CH, or heteroatom;
A=lower alkoxy, lower alkylthio, aryl, subsitituted aryl or fused aryl; and
stereochemistry is variable at the positions marked*
and mixtures thereof.

8. A method according to claim 1 wherein the scalp cleansing composition is dispensed as foam.

9. A method according to claim 1 wherein the scalp cleansing composition is dispensed as an aerosol foam.

10. A method according to claim 1 wherein the scalp cleansing composition is dispensed as a foam with a pump.

11. A method according claim 1 wherein the conditioning shampoo composition is dispensed as a foam.

12. A method according to claim 1 wherein the conditioning shampoo composition is dispensed as an aerosol foam.

13. A method according to claim 1 wherein the conditioning shampoo composition is dispensed as a foam with a pump.

14. A method according to claim 8 wherein the scalp cleansing composition has a zero shear viscosity measures at 0.01 Pa s of less than about 15,000 Pa s.

15. A method according to claim 8 wherein the scalp cleansing composition has a zero shear viscosity measures at 0.01 Pa s of less than about 10,000 Pa s.

16. A method according to claim 8 wherein the conditioning shampoo composition has a zero shear viscosity measures at 0.01 Pa s of less than about 15,000 Pa s.

17. A method according to claim 8 wherein the conditioning shampoo composition has a zero shear viscosity measures at 0.01 Pa s of less than 10,000 Pa s.

18. A method according to claim 1 wherein the scalp cleansing composition and conditioning shampoo composition are included in a single package having a dual chamber.

19. A method according to claim 1 wherein the scalp cleansing/conditioning shampoo composition comprises a benefit agent selected from the group consisting of an anti-dandruff agent, anti-fungal agent, anti-itch agent, anti-bacterial agent, anti-microbial agent, moisturization agent, anti-oxidant, vitamin, lipid soluble vitamin, chelant, perfume, brightener, enzyme, attractant, dyes, pigment, bleach, and mixtures thereof.

20. A method according claim 19 wherein the anti-dandruff agent is zinc pyrithione.

21. Instructions for complying with hair washing and conditioning method of claim 1:
1. Wet hair thoroughly;
2. Position the applicator in direct contact with the scalp and dispense shampoo (cleansing product) directly to the scalp and root;
3. Continue application until entire surface area of the scalp and root are covered;
4. Massage into roots and scalp. Avoid/minimize spreading it onto length and ends;
5. Apply conditioner product to length and ends of the hair and
6. Rinse thoroughly

22. A kit of products comprising 1 or more containers of a scalp cleansing composition, 1 or more containers of a conditioning shampoo composition or a conditioner composition and usage instructions, wherein the scalp cleansing composition includes an attached or detached applicator with a single or plurality of outlets that extend more than 1 cm from its base and/or wherein the instructions suggest zone specific application.
